(19) United States
(12) Patent Application Publication
(10) Pub. No.: US 2026/0363684 A1
(43) Pub. Date: Nov. 19, 2026

(54) INGESTIBLE CAPSULE WITH A THERMOMECHANICAL BIOPSY SYSTEM

(71) Applicant: UNIVERSITY OF MARYLAND, COLLEGE PARK, College Park, MD (US)

(72) Inventors: Michael Straker, College Park, MD (US); Joshua Levy, College Park, MD (US); Reza Ghodssi, College Park, MD (US); Luke A. Beardslee, College Park, MD (US); Justin Stine, College Park, MD (US)

(21) Appl. No.: 19/679,914

(22) Filed: May 18, 2026

Related U.S. Application Data
(60) Provisional application No. 63/807,430, filed on May 16, 2025.

(51) Int. Cl. A61B 10/04; A61B 10/02

(52) U.S. Cl. CPC A61B 10/04; A61B 10/0233; A61B 2010/0208; A61B 2562/162

(57) ABSTRACT

Systems and methods for tissue sampling from the GI tract using a device structured within an ingestible capsule. The device is biocompatible, swallowable, and contains internal and external components including a power source—as well as tailored materials and composition—to allow for autonomous or semi-autonomous (that is, performed substantially without outside control and/or without direct human control) biopsy inside the GI. The mechanism of actuation of the biopsy module of the device may be passive (e.g., operated based on mechanical energy stored within a biopsy module) or active (e.g., electromechanical, driven by mechanical force generated from an electrical input to a motor or similar mechanism).

### CROSS-REFERENCE TO RELATED APPLICATIONS

This patent application claims priority from and benefit of U.S. Provisional Patent Application No. 63/807,430 filed May 16, 2025. The disclosure of which is incorporated by reference herein.

### STATEMENT OF GOVERNMENT SUPPORT

This invention was made with government support under ECCS1939236 awarded by the National Science Foundation. The government has certain rights in the invention.

### RELATED ART

Gastrointestinal (GI) disorders affect millions of people worldwide, often requiring diagnostic procedures to identify and monitor various conditions. Traditional methods for examining the GI tract, such as endoscopy (including endoscopic biopsy), for example, can be invasive and may not provide access to certain regions of the intestines. Additionally, these procedures typically require sedation and specialized medical facilities, limiting their accessibility and frequency of use.

Ingestible devices have emerged as a potential solution for non-invasive GI tract examination and sampling. These devices can traverse the entire digestive system, potentially reaching areas that are difficult to access with conventional tools. However, existing ingestible devices (such as endoscopic ingestible devices) face challenges in precisely targeting specific locations for tissue sampling—and require real-time external monitoring and are operator dependent. Furthermore, the power requirements and mechanical complexity of the current designs limit their effectiveness and reliability in real-world clinical applications—whether in terms of the overall size of the devices or operations requiring current levels that exceed operational capacity (currents of 60 mA or below) of ingestible sized batteries.

While ingestible devices equipped with optical, chemical, and physiological sensors have become commercially available, there appears to be no commercial ingestible device possessing tissue sampling capabilities (that is, biopsy-enabled): discussed in related art thus far biopsy ingestible device prototypes have failed to takeoff due to the reliance on external operation (such as the use of magnetic magnet-stimulated mechanisms that open and close apertures of tissue sample reservoirs), impractically high-power requirements of actuation mechanisms not attainable by the available ingestible-sized batteries, of inability to collect tissue samples of suitable quality for practically useful characterization. Similarly, related art has not yet addressed the question of whether an ingestible device can operate autonomously—that is, the problem of providing the ingestible device with a capability to act independently without outside control (in particular, without direct human control) remains of practical interest. Notably, at least for the purposes of the present disclosure, the utilization of magnetic field and/or magnet-stimulated mechanisms for operation of the ingestible device is not considered to be the autonomous operation.

And yet, there remains a persisting need in a reliable, low-power consuming methodology of collection GI tract tissue sample(s) for evaluation by a pathologist to reach a definitive diagnosis (to reveal microscopic structural abnormalities such as mucosal degradation, dysplasia, and immune cell infiltration, for example).

### SUMMARY

Embodiments of the invention provide an autonomous wireless ingestible device structured to operate, with the use of a local power source, to obtain tissue samples from targeted remote GI regions with the use of a low-power actuator that eliminates the need for high-powered magnets and actuators of related art.

In particular, embodiments provide an ingestible device—for gastrointestinal (GI) tissue sampling—that includes a housing; a power source disposed within the housing; and a biopsy module that contains an actuator mechanism operably coupled to the power source and a biopsy collection member that has a cavity or chamber defining an internal volume and that is actuatable by the actuator mechanism to have the cavity reversibly extend outside of the housing, where the device is operable without direct human control. Notably, the device does not involve magnets and/or magnet-stimulated mechanisms. At least in one implementation, the device may be equipped with a trigger mechanism to trigger the actuation mechanism, and the trigger mechanism may include a bioimpedance sensor; a wireless communication system; a pH sensor; an electrochemical sensor; a timer; and a specific biomarker sensor and/or a temperature sensor. (Optionally, the trigger mechanism may include the electrochemical sensor operable to monitor a characteristic of the GI tract tissue in real time and to determine an area of interest for monitoring the characteristic based on electrochemical signals registered inside the GI tract.)

In at least one specific implementation, the actuator mechanism includes a rotational member operated in response to power delivered from the power source (the rotational member being configured to move the biopsy collection member into contact with the GI tract tissue, and/or an initiating member configured to move from a detained position to a released position to apply a rotational force to the rotational member, and/or a cam of a cam-and-follower mechanism to convert a rotational motion into a linear displacement of the biopsy collection member, where the biopsy collection member is structured as a follower of the cam-and-follower mechanism while the housing is configured as a frame and a guide of the cam-and-follower mechanism). Alternatively or in addition, the actuator mechanism may contain a gear train operable to transmit torque from a structural element of the device to the biopsy collection member after status of the structural element has been modified by the power source. The biopsy collection member may be dimensioned as i) a biopsy punch having a cylindrical shape or as ii) a rotatable cavity, to have the internal volume of approximately 1 mm³ to approximately 5 mm³ to hold a sample of the GI tissue sufficient in size for clinical diagnostic testing, and have a bladed rim. Alternatively or in addition, the actuator mechanism may be structured to include a spring, a microheater, and a thermally responsive adhesive. (Here, the spring is placed with one end fixed to the housing and another end attached to a rotatable component of the device; the microheater is thermally coupled to the thermally responsive adhesive that restrains the spring in a loaded state and releases the spring to apply rotational force to the rotatable component of th device to drive the biopsy collection member when the adhesive is molten or softened by heat energy from the microheater. In one specific case, the spring may be connected to and retained with a gear that is directly bonded to a fixed element of the actuation mechanism with the thermally responsive adhesive.)

Embodiments additionally provide an autonomous ingestible device for gastrointestinal (GI) tissue sampling, which device includes a housing; an on-board power source contained within the housing; a moveable apparatus coupled with the housing and having a portion that is reversibly repositionable from a retracted position inside the housing to an extended position outside the housing upon autonomous activation by power delivered from the power source; a thermally responsive adhesive disposed within the housing and configured to retain the biopsy collection member in the retracted position; a spring disposed within the housing and operatively connected to the portion of the movable apparatus, where the spring is in a loaded state when the portion is in the retracted position; and a heater electrically coupled to the power source and disposed adjacently to the thermally responsive adhesive. The reversibly repositionable portion of the moveable apparatus may include microscale biomimetic tissue collector structures on its inner surface.

Embodiments of the invention further provide a method for autonomously collecting gastrointestinal (GI) tissue by performing at least the following actions: a) actuating a rotational motion of a driving mechanism of an ingestible device that contains a power source inside a capsule housing of the device by delivering a power from the power source to a thermally-responsive retaining element mechanically connected with the driving mechanism; and b) with said rotational motion, protruding a biopsy collection member from inside the housing to outside the housing to intersect the GI tissue. In one specific implementation, the step of actuating may include applying a rotational force to the driving mechanism with an energy-storing element of the ingestible device upon releasing the energy-storing element from a loaded state.

### BRIEF DESCRIPTION OF THE DRAWINGS

The invention will be more fully understood by referring to the following Detailed Description of Specific Embodiments in conjunction with the Drawings, of which:

FIGS. 1A, 1B illustrate, in exploded isometric views, related non-limiting embodiments of a device of the invention with some internal components exposed for easy viewing.

FIG. 1C illustrates an embodiment of on-board electronic circuity with which the device of FIGS. 1A, 1B may be equipped.

FIGS. 2A, 2B, 2C, 3A, 3B, 3C, 3D, 4A, 4B, and 4C aggregately depict the structure and principle of operation of one embodiment of the biopsy module equipped with a biopsy collection member that is structured as a punch and driven by an actuator mechanism.

FIGS. 5A, 5B, and 5C illustrate alternative structures of a biopsy punch of an embodiment of the biopsy module structured according to the idea of the invention.

FIGS. 6A, 6B illustrate the results of COMSOL simulations that depict the rotation of the cam and resulting translations of the follower (employed to reposition the biopsy punch such as that of embodiments of FIGS. 5A, 5B, 5C), along with the vertical displacement of the follower over time.

FIGS. 7A, 7B, 7C, 7D, and 7E illustrate a particular implementation of a low power actuation mechanism for on-command protrusion and retraction of a biopsy collection member from an embodiment of the ingestible capsule structured according to the idea of the invention. FIG. 7A: a biopsy module with an actuation mechanism; FIG. 7B: an embodiment of the biopsy punch; FIG. 7C: an internal rotary track (cam profile) featuring a paddle rotated with the use of a spring element of the actuator mechanism; FIG. 7D: illustration of a preloaded spring element fastened to a peg of the biopsy module fastened to a peg of the module housing and the rotary track paddle; FIG. 7E: the resistive microheater adhered to the rotary track with a thermally responsive material (e.g., polycaprolactone) holding the rotary track in place until activation;

FIG. 7F (sub-figures 1, 2, 3, and 4): steps of activation of the biopsy collection member.

FIGS. 8A, 8B, 8C, 8D, 8E, and 8F schematically illustrate details and operation of an ingestible capsule device containing a biopsy module that is equipped with a scoop-shaped rotatable biopsy collection member and an associated gear-train-based activation mechanism of a related embodiment of the invention. FIG. 8A: a CAD rendering depicting the ingestible capsule device containing the biopsy module with wireless capsule electronics inside a 3D-printed shell compartment. FIG. 8B: a 2D schematic of contents of the integrate capsule device. FIG. 8C: photograph of the embodiment showing its final dimensions. FIG. 8D: Depiction of the internal gear mechanism. FIG. 8C: Depiction of integrated Au-heater element for triggering of the actuation mechanism of the biopsy module. FIG. 8F: general structure and technical details of operation of the biopsy module.

FIGS. 9A, 9B, 9C, 9D provide additional illustrations describing mechanical cooperation among the constituent components of the biopsy module of FIGS. 8A-8C and its operation.

FIG. 9E illustrates the process of fabrication of at least some of components of the embodiment of FIGS. 8A, 8B, 8F.

FIGS. 9F-1, 9F-2, and 9F-3 illustrates the results of characterization of an embodiment of the biopsy collection member of the module 800 fabricated using hybrid 3D-printing techniques combining printing techniques. FIG. 9F-1 presents an image depicting the experimental setup in which the scoop (biopsy collection member of the biopsy module) is driven into and subsequently pulled out of an agarose phantom. (scale bar: 6 mm). FIG. 9F-2 includes plots illustrating penetration and retraction force characterizing such biopsy collection member measured by the force tester on the agar phantoms of varying stiffness. FIG. 9F-3: A bar graph summarizing the results of the characterization (n=3). The y-axis represents the log of the Force in mN.

FIG. 10 illustrates a non-limiting implementation of the overall apparatus or system 1000 governing and/or utilizing the operation of an embodiment of the ingestible capsule device.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. Specific embodiments are depicted in the figures with the understanding that the disclosure is intended to be illustrative and are not intended to limit the scope of invention the implementations of which are described and illustrated herein.

### DETAILED DESCRIPTION

The ingestible biopsy-performing device described herein is structured to be used for gastrointestinal tissue sampling in various living subjects, including but not limited to humans, animals, and potentially other organisms with a digestive tract. The dimensions of the device associated with it's being “ingestible” may vary depending on the subject for which the device is intended. For human subjects, an ingestible device should preferably fit in a capsule-like housing with a diameter of around 11 mm or less and a length of around 33 mm or less. However, these dimensions may be somewhat adjusted for different subjects or applications. For instance, larger animals may accommodate devices with larger dimensions, while smaller animals or specialized applications may require more compact designs. The scalability of the device's components allows for adaptation to various subject sizes and physiological requirements while maintaining the core functionality of non-invasive gastrointestinal tissue sampling.

Interchangeably, an embodiment of an ingestible biopsy-capable device discussed below—designed to traverse the GI tract and perform targeted GI-tract tissue sample)—may be referred to as an “ingestible capsule device” (or “capsule”, for short), considering a preferably capsule-like housing in which the contents of the device are intended to fit.

### I. Examples of an Ingestible Capsule Device and Constituent Components

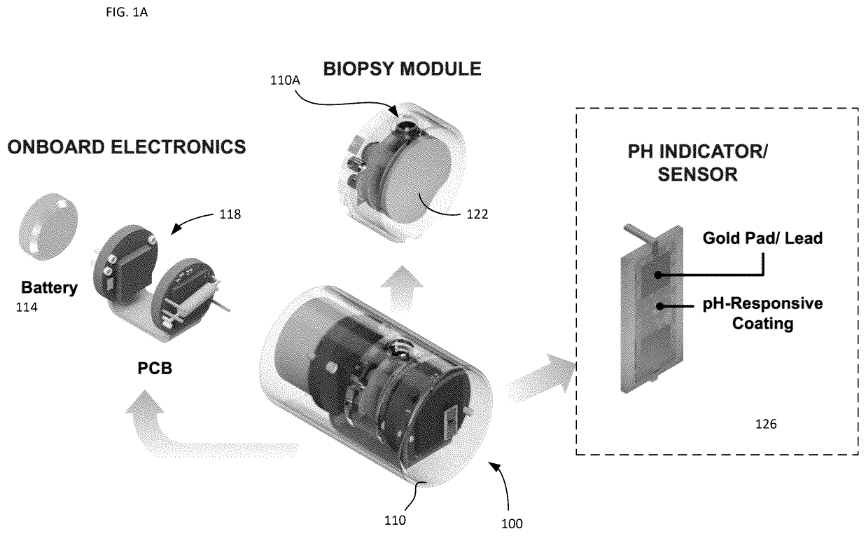

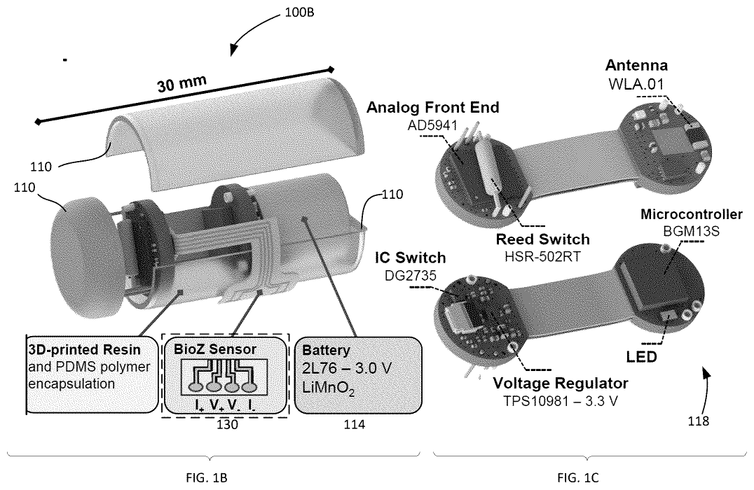

FIGS. 1A, 1B present schematics of related embodiments 100A, 100B of the ingestible capsule device structured, according to the idea of the invention, to include (an optionally multi-piece) capsule-like housing or shell 110 containing the appropriately dimensioned power source 114, control systems and electronics 118 (generally including a microcontroller and a communication module) for operation and data transmission, and a biopsy module 122 containing a biopsy collection member (not specifically shown) judiciously spatially coordinated with a corresponding aperture 110A in the housing 110 (through which the biopsy of the target GI tissue is carried out). FIG. 1B does not illustrate the biopsy module.

The biocompatible housing 110 is designed to traverse the gastrointestinal tract, and may be substantially cylindrically shaped with rounded ends. In one implementation, the housing 110 is fabricated from medical-grade polymers such as polyethylene, polypropylene, or polytetrafluoroethylene (PTFE) with dimensions ranging from about 10 mm to about 30 mm in length and from about 5 mm to about 15 mm in diameter, with wall thickness of between about 0.5 mm and about 2.0 mm. In some cases, the housing may include alignment features such as internal ribs or slots with dimensional tolerances of ±0.05 mm (not shown).

A power source 114 disposed within the housing 110 is configured to provide energy for the operations of the components of the device 100A, 100B and may take the form of a miniature battery, as shown (such as a silver oxide or lithium coin cell battery). In some cases (not illustrated here), the power source 114 may utilize energy harvesting techniques to extend operational lifetime, for instance by incorporating a piezoelectric element to convert mechanical energy from GI motility into electrical energy. In one case, the power source may deliver between 1.5 V and 3.0 V with a capacity ranging from 30 mAh to 150 mAh, depending on the specific implementation requirements (for instance, the power source may be implemented using lithium manganese dioxide coin cells with a nominal voltage of 3.0 V and capacity of 30 mAh). The power source 114 may be electrically coupled to power management circuitry (which may be part of onboard electronics 118) including low-dropout regulators to maintain stable voltage levels for electronic components, and/or the power source may incorporate overcurrent protection circuitry to prevent damage from short circuits.

On-board electronic circuitry 118—a specific example of which is shown in FIG. 1C—include a wireless printed circuit board (PCB) for data transmission and control (utilizing, in one specific case, low-power wireless protocols such as Bluetooth Low Energy, BLE, or ZigBee to communicate with external receivers). The PCB may incorporate microcontrollers such as ARM Cortex-M series processors to manage device functions and process sensor data. For example, the PCB may be implemented using a four-layer FR-4 substrate with a thickness of 0.8 mm and copper traces of 1 oz weight (35 μm thickness). In some examples, the PCB may include a system-on-chip with an ARM Cortex-M4F processor operating at about 16 MHz to about 64 MHz, 512 KB flash memory, and 64 KB RAM. The wireless communication may operate in the 2.4 GHz ISM band with a maximum transmit power of 4 dBm and receiver sensitivity of −96 dBm. The PCB may incorporate power management integrated circuits such as buck converters to efficiently regulate voltage from the power source to the required levels for digital and analog circuitry. The PCB layout may utilize controlled impedance traces with widths of 0.15 mm to 0.25 mm and spacing of 0.15 mm to 0.3 mm for high-frequency signal routing. For example, the PCB may incorporate ground and power planes on inner layers. In some cases, the PCB may be coated with a conformal coating such as acrylic or silicone with a thickness of 25 μm to 75 μm. Specific constituent components of the electronic circuitry 118 illustrated in FIG. 1C are but examples only and are non-limiting.

In certain embodiments, an embodiment of the ingestible capsule may be complemented with an anchoring mechanism (not shown in FIGS. 1A-1C) to maintain positional stability during the biopsy (tissue acquisition) and/or one or more electrochemical sensors to detect biochemical signals for site selection. To this end, an embodiment of the ingestible capsule may include a passive anchoring mechanism similar to those disclosed in US 2022/0401024A1, the disclosure of which is incorporated by reference herein. The anchoring mechanism may contain microdarts or barbed legs deployable from the capsule housing. These anchoring elements may be biased by internal springs and held in a compressed state until the capsule has reached a desired location in the GI tract. Once at a location of interest, a mechanical actuation caused by an electrical signal or sensor data reflective of environmental conditions—such as changes in pH, bioimpedance, temperature, or contact pressure—cause the anchors to deploy so as to removably lodge the capsule in the surrounding tissue. The passive anchoring ensures minimal power consumption and is particularly suited for prolonged dwell times at a sampling site. Alternatively, an active anchoring mechanism may be employed in which anchoring components are held in place by a thermal-release element. (In this configuration, a particular internal resistive heater present within the housing may be energized by the capsule's onboard power source 114 to melt a restraining material that otherwise restrains cantilever arm, which is released upon melting of the restraining material to deploy anchor(s) into the surrounding GI tissue to affix the capsule at a location of interest. In an alternative embodiment, the active anchoring mechanism may include a mechanically actuated pin release system. For example, the ingestible capsule device may contain a retention pin that restrains a spring-biased anchor arm. When a dedicated internal actuator—such as a microservo or another electromechanical actuator—removes or retracts the pin, the anchor arm swings outward, embedding microdarts into the intestinal wall. The actuation may be initiated based on an internal timer, sensor signal, or wireless command. Understandably, this active approach to anchoring the ingestible capsule within the GI tracts allows for substantially precise control over the anchoring timing and location based on sensor input or preprogrammed logic.

Optionally (as indicated with dashed lines), a particular specific embodiment of the ingestible capsule may additionally or in the alternative contain one or more electrochemical sensors embedded in or near the outer surface of the housing (as shown in FIGS. 1A, 1B—sensors 126, 130. These sensors are configured to detect biomarkers indicative of inflammation or other gut disorders, including by sensing GI environmental conditions which may include, but not limited to pH, redox potential, or concentrations of specific hormones or metabolites. In an embodiment equipped with such sensors 126, 130. Such embodiments may be configured to process the sensor output by onboard electronics 118 and, upon detection of a threshold signal, initiate at least one of anchoring of the ingestible capsule and/or biopsy sequence. Such optional electrochemical sensing functionality may be implemented using microfabricated electrodes integrated into the capsule's surface, similar to those disclosed US Patent Application Publication 2023/0404428A1, the disclosure of which is incorporated herein by reference.

### (I.1) Non-Limiting Examples of Embodiments of a Biopsy Module

### General

Regardless of the specifics of a particular implementation, the biopsy module 122 is generally structured as a low power, closed-loop operation module that includes a structural member movably coupled to the housing 110 and repositionable—linearly or rotationally—between a retracted position and an extended position. The operation of structural is controlled by an embodiment of an actuating mechanism (interchangeably—an actuator mechanism or actuator) that is operably coupled to the power source of the capsule and that generally includes a spring element and a thermally responsive/thermally activated (meltable) adhesive—for example, glue or polymer—that retains the structural member in the retracted position and, when melted, causes the structural member to change its position between the retracted and extended positions with the help of the spring element. The change of a state of the thermally responsive adhesive is preferably controlled and/or activated with the use of a microheater of the actuator mechanism

### (A) Embodiment Employing a Biopsy Punch and Corresponding Actuator

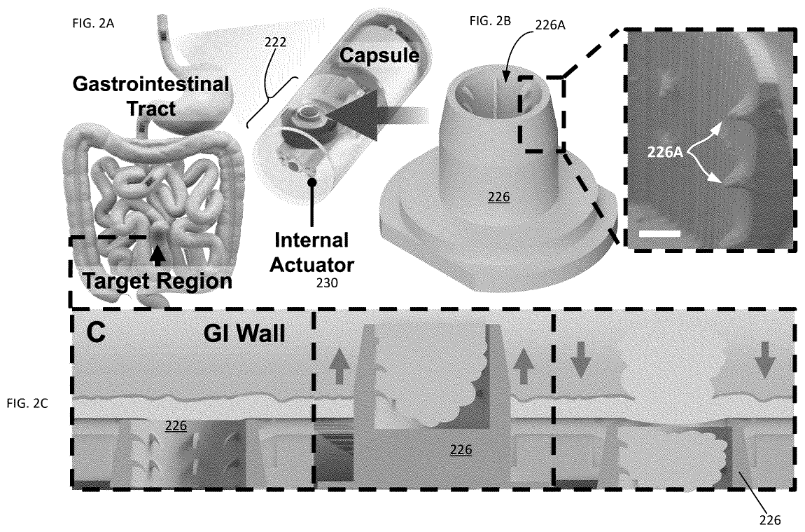

FIGS. 2A, 2B, 2C, FIGS. 3A, 3B, 3C, and 3D, and FIGS. 4A, 4B, and 4C aggregately depict the structure and principle of operation of one embodiment of the biopsy module 222 that employs a biopsy collection member 226 structured as a punch (shaped to define an internal volume 226A dimensioned to store a GI tract tissue obtained during the biopsy procedure) and driven by an actuator mechanism 230.

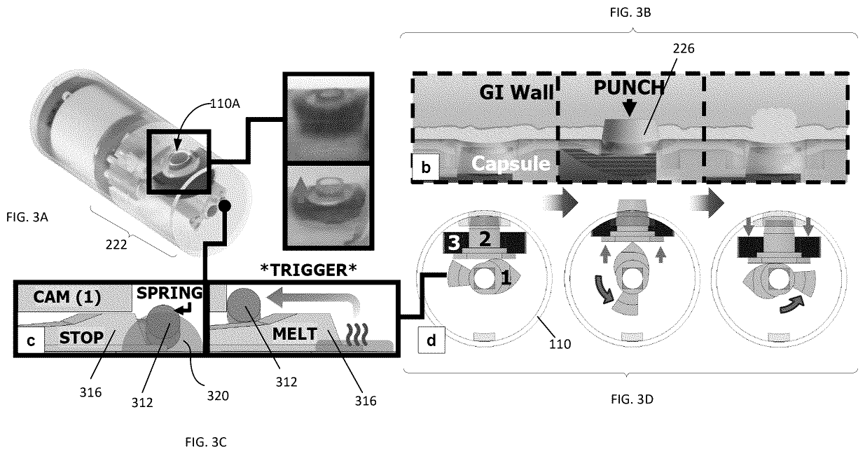

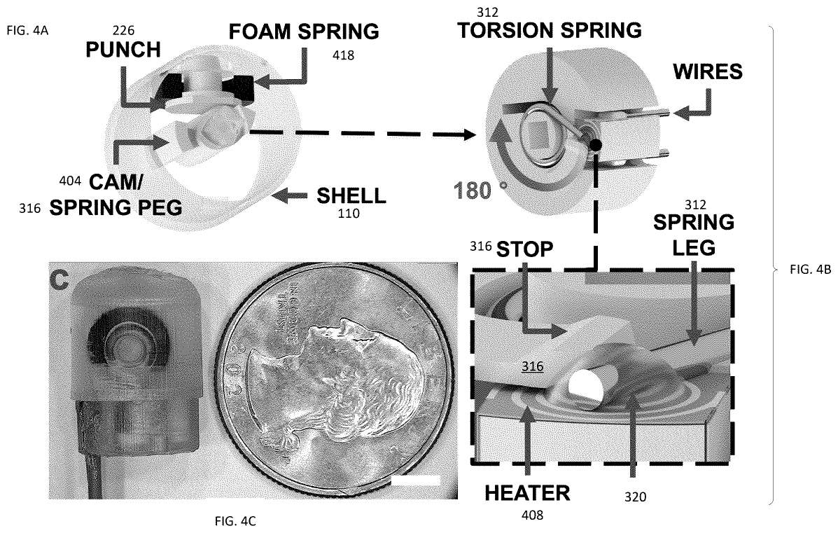

As schematically shown in FIG. 2A, the ingestible capsule with the integrated biopsy module 222 travels to the target region (for example, in a small intestine), where the onboard electronics 118 of the device trigger the actuator 230—here, employing a cam (identified in FIG. 3D as “1” and in FIG. 4A as 404), a spring (312 in FIG. 3C), a spring peg 316, a microheater 408, and thermally responsive adhesive 320

The power source 114 energizes a microheater element 408 that is positioned on a mounting structure within a trigger portion of the ingestible capsule. The microheater is thermally coupled to and in contact with the meltable adhesive material 320 that initially restrains a torsion spring 312 in a loaded state. The torsion spring 312, fabricated e.g. from a stainless-steel wire with a diameter of 0.2-0.5 mm, is configured with one end fixed to the housing and the other end attached to the spring peg 316 while in a loaded state. When the microheater 408, electrically connected with the power source 114, is activated by the power source the microheater releases thermal energy sufficient to melt the adhesive material 320, releasing the torsion spring 312 which then applies force to the cam and drives the rotation of the cam. The cam, which may be fabricated from medical-grade polymers such as polyoxymethylene (POM), features a precisely designed profile that translates rotational motion into linear displacement of a biopsy punch 226 (denoted as “2” in FIG. 3D) that is configured as a follower in a cam-and-follower mechanism of FIG. 3D. The biopsy punch 226 extends through the housing opening 110A towards the GI wall to collect a tissue sample (see FIGS. 2C, 3B). A foam spring element (shown as “3” in FIG. 3D and as 418 in FIG. 4A), positioned around the follower/punch 226, provides a return force to retract the biopsy punch back into the housing 110 after tissue collection. This integrated mechanism enables precise control of the sampling process while maintaining a compact form factor suitable for an ingestible device.

In at least one specific case, a biopsy punch is shaped substantially as a cylinder (as shown in FIG. 2B) defining an internal volume 226A for storing GI tract tissue obtained during sampling. In some cases, the cylindrical punch may have an inner diameter of about 1 mm to about 3 mm and a length of about 5 mm to about 10 mm, thereby providing sufficient volume for clinical diagnostic testing of the sampled tissue. For example, the internal volume 226A is dimensioned to house about 5 mg to about 20 mg of sample tissue, which—as the skilled person will readily appreciate—is adequate for histological analysis or polymerase chain reaction (PCR) testing. (For this purposes, in one example, the volume of the cavity of the biopsy collection member may be from approximately 1 mm³ to about 5 mm³.) The cylindrical punch 226 may be fabricated from medical-grade stainless steel such as 316L with a wall thickness between about 0.1 mm and about 0.3 mm. In some examples, the leading edge of the punch may be precision-ground (bladed) to a cutting angle between 15-30 degrees. The internal surface of the punch 226 may be treated with a low-friction coating such as parylene-C with a thickness of about 2 to about 5 μm. The punch may additionally incorporate a spatially tapered design with the diameter decreasing by about 0.1 mm to about 0.2 mm from the leading edge to the base to facilitate the retention of tissue once collected.

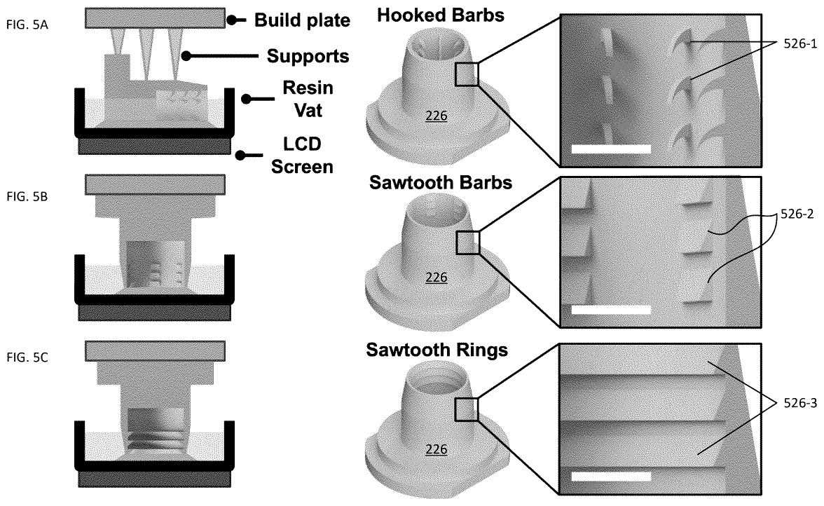

Alternatively, as is schematically shown in illustrations of the first column of FIGS. 5A, 5B, 5C, a body of the biopsy collection member may be fabricated using hybrid 3D printing techniques combining liquid crystal display (LCD) printing and direct laser writing. For example, the base structure of the biopsy collection member 226 may be printed using an LCD 3D printer with a resolution of 50 microns, while finer features such as microscale barbs may be added using a two-photon polymerization direct laser writing system with a resolution down to 200 nanometers. The LCD 3D printer may utilize a photopolymer resin such as methacrylate-based resins with biocompatible additives. The direct laser writing system may employ a femtosecond laser with wavelength of 780 nm and pulse duration of 100 femtoseconds. In some examples, the hybrid fabrication process may include post-processing steps such as ultraviolet curing at 405 nm wavelength for 10 minutes followed by isopropyl alcohol washing to remove uncured resin residues.

In further reference to FIG. 2B, to improve tissue sample retention the biopsy collection member (punch 226, in this example) may incorporate biomimetic tissue collector structures 226B on the inner surface of the biopsy collection member. As illustrated in FIGS. 5A, 5B, 5C and, the structures 226B may take various forms inspired by biological mechanisms, such as hooked barb structures 526-1 of FIG. 5A, shaped similarly to the proboscis of intestinal hookworms and fabricated with a curve radius of 50-100 microns and a tip diameter of 5-10 microns, for example. The hooked barbs 526-1 may be arranged in a helical pattern along the inner surface of the cylindrical punch 226 with a pitch of 200-400 microns and angular spacing of 30-60 degrees. For example, the barbs 526-1 may be fabricated using direct laser writing in a photosensitive polymer such as IP-Dip with a Young's modulus of 2 . . . 3 GPa. In some cases, the barbs 526-1 may be metallized with a thin layer of gold or platinum with a thickness of about 50 nm to about 100 nm using sputter deposition. The density of barbs 526-1 may range from 10-50 per square millimeter, with higher densities near the leading edge of the punch.

Alternatively, the biopsy collection member may feature sawtooth barb structures 526-2 inspired by the teeth of intestinal hookworms, see FIG. 5B For example, these sawtooth barbs may be arranged in rows with a pitch of 100-200 microns and a tooth height of 50-100 microns. The sawtooth profile may be designed with an angle of about 30 degrees to about 60 degrees. The sawtooth barbs 526-2 may be fabricated using micro-electrical discharge machining (μEDM) with electrode diameters of 50-100 microns and discharge energies of 10-50 μJ. In some examples, the sawtooth structures may be arranged in a staggered pattern across multiple rows, with row-to-row offsets of about 50 microns to about 100 microns. The barbs 526-2 may be fabricated from hardened stainless steel with a Rockwell hardness of 45-55 HRC. For instance, the leading edges of the sawtooth structures may be further enhanced through electrochemical polishing using a solution of phosphoric and sulfuric acids with current densities of 5-10 A/dm² for 30-60 seconds to achieve edge radii of 1-5 microns. The sawtooth barbs 526-2 may incorporate asymmetric profiles with steeper angles (50-70 degrees) on the trailing edge and shallower angles (20-40 degrees) on the leading edge.

In some implementations—such as that of FIG. 5C, for example—the biopsy collection member 226 may include sawtooth ring structures 526-3 on the inner surface thereof. These ring structure may be positioned circumferentially within the cylindrical punch 226, with each ring featuring sawtooth projections facing inward. For instance, the sawtooth rings may be spaced at intervals of 0.5-1 mm along the length of the punch, with tooth heights ranging from 50-200 microns depending on the specific tissue targeting requirements. The sawtooth ring structures 526-3 may be fabricated using precision micro-molding techniques with mold inserts created through micro-electrical discharge machining or direct laser writing. (For example, the rings may be injection molded using biocompatible polymers such as medical-grade polyetheretherketone (PEEK) with melt temperatures of 360-380 °C and mold temperatures of 170-190 °C.) Alternatively, the ring structures 526-4 may be fabricated as separate components and then assembled into the cylindrical punch 226 using interference fits or biocompatible adhesives such as cyanoacrylates. These sawtooth ring structures may incorporate varying tooth geometries along the length of the punch, with more aggressive profiles (tooth height to width ratios of 1:1) near the opening and less aggressive profiles (ratios of 1:2 or 1:3, for example) deeper within the punch.

Referring again at least to FIGS. 3C and 4B, embodiments of the actuation mechanism that utilize a torsion spring (coupled to a microheater for triggering and held in place by a meltable polymer that is released by operation of the microheater) may employ a torsion spring fabricated from stainless steel wire with a diameter ranging from 0.2 mm to 0.5 mm and a spring constant between 0.1 N·mm/degree and 0.5 N·mm/degree. In some examples, the torsion spring may be held in place by a low-melting point thermoplastic polymer such as polyethylene glycol (PEG) with a molecular weight between 1000 and 4000 g/mol, which melts at temperatures between 35 °C and 60 °C. The torsion spring may be manufactured using precision wire forming techniques with coil diameters between 1.5 mm and 3.0 mm and leg lengths of 3 mm to 8 mm. For instance, the spring may be heat-treated at temperatures between 400 °C and 500 °C to relieve internal stresses and improve elastic properties. In some cases, the spring may be coated with a thin layer of nickel or chromium with a thickness of 2 μm to 5 μm to enhance corrosion resistance in the gastrointestinal environment.

The microheater used for triggering may be implemented as a thin-film resistive element deposited on a polyimide substrate, with dimensions of approximately 2 mm×2 mm and a resistance from about 10 Ω to about 50 Ω. In one case, the microheater is fabricated using platinum traces with a thickness from about 100 nm to about 200 nm deposited on a 25 μm thick polyimide film using electron beam evaporation and patterned using photolithography techniques. In one implementation, the microheater incorporates a serpentine pattern with trace as wide as 50 μm to 100 μm and spacing of 100 μm to 200 μm to achieve uniform heat distribution across the active area. The microheater may include contact pads fabricated from gold with dimensions of 0.5 mm×0.5 mm and a thickness of 200 nm to 500 nm to facilitate reliable electrical connections. Optionally, the microheater is encapsulated with a protective layer of silicon dioxide or silicon nitride with a thickness of 300 nm to 800 nm. Embodiments of the microheater were operated at a power level between about 50 mW and about 150 mW to generate heat sufficient for melt the employed polymer within 1 to 3 seconds. Optionally, the microheater may incorporate a temperature sensor (such as a platinum resistance temperature detector, RTD) to provide feedback control of the heating process.

Embodiments of the punch-actuating mechanism that include a dedicated spring (for example—a foam spring 428 of FIG. 4A, positioned to facilitate the return of the biopsy punch back into the housing 110 after sampling the tissue) may employ the foam spring fabricated from open-cell polyurethane foam with a density of 20 kg/m³ to 40 kg/m³ and a compression set of less than 10%. The foam spring may be cut into a cylindrical shape with a diameter of 4 mm to 6 mm and a height of 2 mm to 4 mm, providing sufficient restoring force while maintaining a compact form factor. For example, the foam spring may be manufactured using a water-blown polyurethane formulation with a cell size between 200 μm and 500 μm. The foam may be treated with a silicone coating with a thickness of 5 μm to 20 μm. In some cases, the foam spring may be designed with a central hole or cavity to accommodate the biopsy punch shaft while maintaining uniform compression characteristics. The foam spring may exhibit a compression force of 0.1 N to 0.5 N when compressed to 50% of its original height.

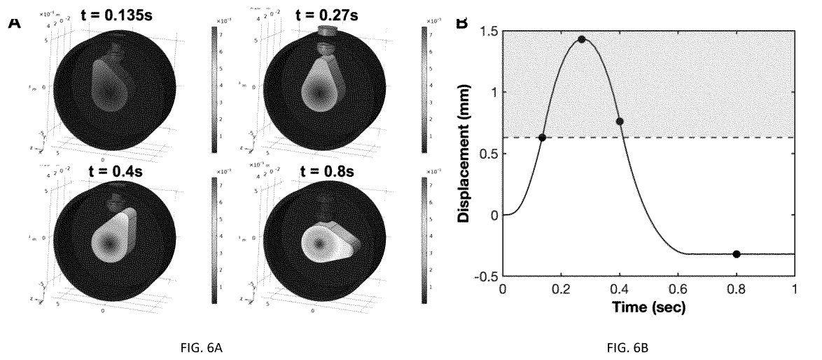

FIGS. 6A, 6B illustrate the results of COMSOL simulations that depict the rotation of the cam 404 and resulting translations of the follower (the punch 226) at 0.135 s, 0.27 s, 0.4 s, and 0.8 s (left), along with the vertical displacement of the follower over time (right). The gray region of FIG. 6B represents the GI wall and indicates anticipated depth of biopsy. The simulations evidenced an estimated displacement of 0.8 mm for the follower out of the capsule shell 110 (through the aperture 110A), which in practice is sufficient to penetrate and collect the mucosal tissue without risk of perforation (as the thickness of the small intestine mucosa and wall are 0.4-1 mm and ~1.5 mm respectively). The cam profile may have a rise of 1 mm to 2 mm over a 90-degree rotation, providing the necessary linear motion for tissue sampling. The cam profile may be devised with a modified sinusoidal displacement curve and fabricated from hardened stainless steel with a surface hardness of 40 HRC to 45 HRC, or from a self-lubricating polymer such as polyetheretherketone (PEEK) with 10% to 15% polytetrafluoroethylene (PTFE) content. In some cases, the follower may incorporate a roller bearing with a diameter of 1.5 mm to 2.5 mm, with the roller fabricated from hardened steel or ceramic materials such as silicon nitride or zirconia.

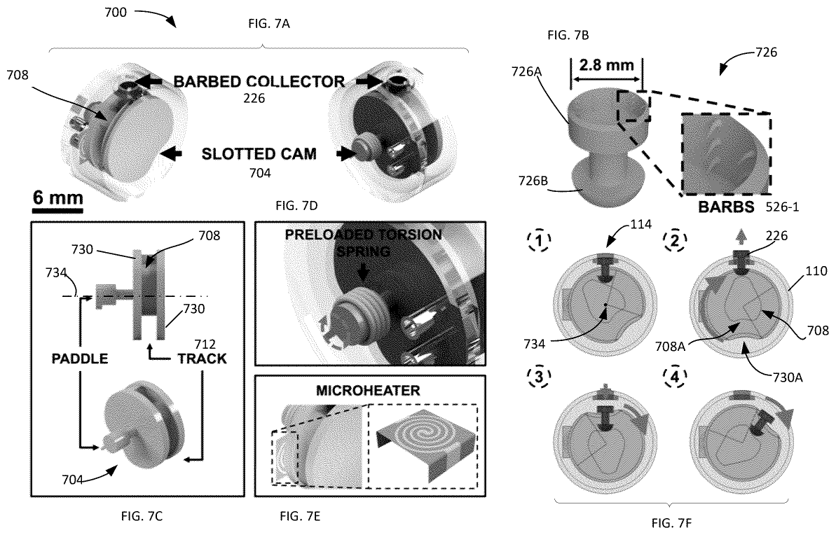

In one particular implementation 700, schematically illustrated in FIGS. 7A, 7B, 7C, 7D, 7E, 7F, the actuator mechanism may include a specifically structured cam component 704, a cam-profile 708 of which is machined into a body of a component having a generally cylindrical outer profile with a diameter of 5 mm to 8 mm such as not only to be monolithic with but also be located in a circumferential slot or trench or track 712 formed in such cylindrical component. (Denoted as “slotted cam” in FIG. 7A). The biopsy punch 726 has an appropriate outer shape that includes a front cup-shaped end 726A (the internal surface of which in this particular non-limiting example is equipped with the hooked barbs 526-1) and a bulb-shaped rear end 726B dimensioned to fit in and substantially frictionlessly move inside the slot track 712 in contact with a surface of the cam-profile 708 upon the rotation of the cam 704. The end surfaces of the substantially parallel to one another walls 730, formed in the body of the cam 704 and defining the slot/track 712, are judiciously profiled to incorporate spatially coordinated with one another indentations or recesses 730A. At the same time, a portion of the cam profile 708 opposing these recesses (and spatially coordinated with these recesses along a direction of an axis that is normal to the axis 734 of the cam 704) is similarly shaped to define a concave portion 708A. The radially-measured separation between the surfaces of the recesses 730A and the surface of the concave portion 708A of the cam profile 708 and the depths of the indentations/recesses 730A in the outer substantially cylindrical edge surface(s) of the cap component 704 are judiciously dimensioned to allow the front, head portion 726A of the punch 726 not protrude beyond the maximum radial extent of the substantially cylindrical body of the cam 704 while accommodating the length of the punch 726 within the trench/slot 712 when, at a particular point of rotation of the cam, the rear end 726B of the punch 726 comes into contact with the concave surface 708A of the cam-profile 708, thereby completely preventing any mechanical interference between the mechanically moveable components of the actuator mechanism 700 during the operation.

At the same time, the sequence of steps of actuation of the biopsy collection member (in FIGS. 7A through 7E: 726) in substantially every implementation involving an actuation mechanism that contains a cam component is illustrated in sequence of sub-figures 1, 2, 3, and 4 of FIG. 7E: 1—resting position with a biopsy punch recessed in the actuator module; 2—the cam rotates, pushing the biopsy collector through the aperture 114 of the housing 110; 3—the biopsy collector is retracted into the housing 114; and 4—the biopsy collector is rotated away from the aperture 114 for sealing.

### (B) Embodiment Employing a Rotatable Cavity and Corresponding Actuator

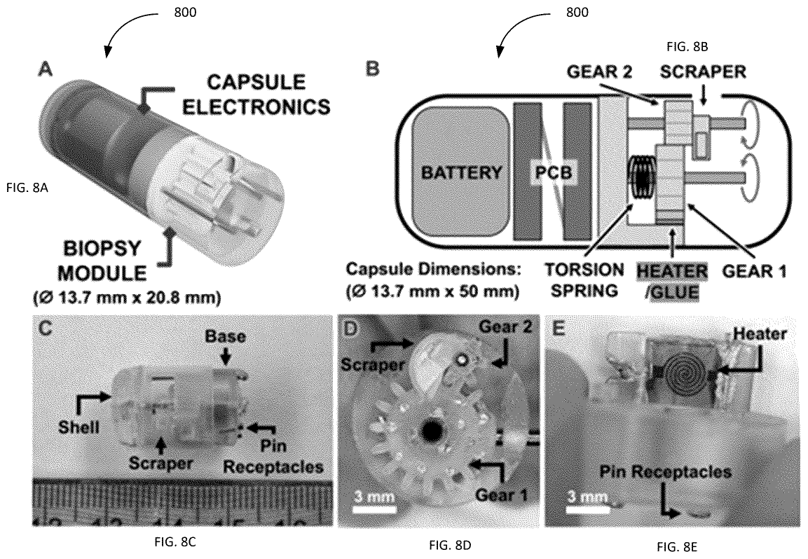

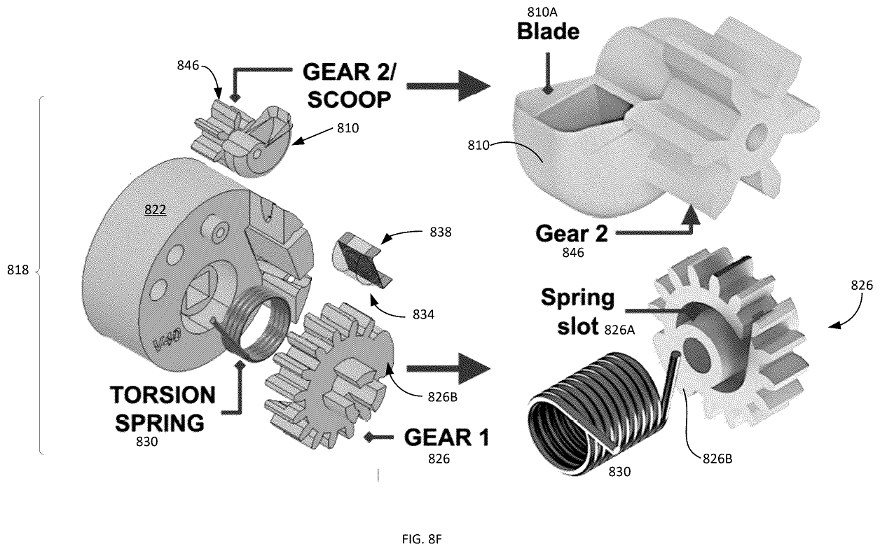

In a related non-limiting embodiment—discussed below in reference to at least FIGS. 8A, 8B, 8C, 8D, 8E, and 8F—the ingestible capsule device 800 features a modular design that contains all actuator and tissue sampling components of the device in a single 3D-printed shell compartment, with electrical connections facilitated by embedded pin receptacles on its base. This allows it to be evaluated and refined independently, while remaining readily configurable for capsule electronics of varying designs. An overview schematic of a fully assembled capsule device 800 is shown in FIG. 8B. The housing capsule contains: an ingestible battery for powering the electronics, a PCB for receiving wireless signals and triggering actuation, and the components of the biopsy collection member and corresponding activation mechanism. The use of flexible polyimide-based heater fabrication, 3D-printed channels for housing electrical interconnections, and a gear assembly that fits within the actuator module footprint enables significant miniaturization of the overall device 800.

Once assembled, the entire device 800 measures about 50 mm in length and about 13.7 mm in diameter, with the biopsy module (FIG. 8C) occupying only about 42% of the total volume and the remainder occupied by the electronics and battery. The module base serves multiple functions: (i) providing channels for electrical interconnects between the heater and pin receptacles, and (ii) acting as a mechanical anchor for the module, ensuring stability during operation. The gear-train system, schematically shown in FIG. 8D and discussed below in reference to FIG. 8F was selected for its flat, compact profile and ability to efficiently transfer torque from the spring to the tissue scraper throughout actuation. The rotational motion allows the scraper (biopsy collection member) to translate out of the capsule enclosure, collect a sample, and return for storage. The ~70-90 Ω Au-resistive heater (FIG. 8E) is made flexible, allowing it to be nested in a compact slot in the base. The heater is electrically connected to the pin receptacles through silver epoxy-filled conductive channels.

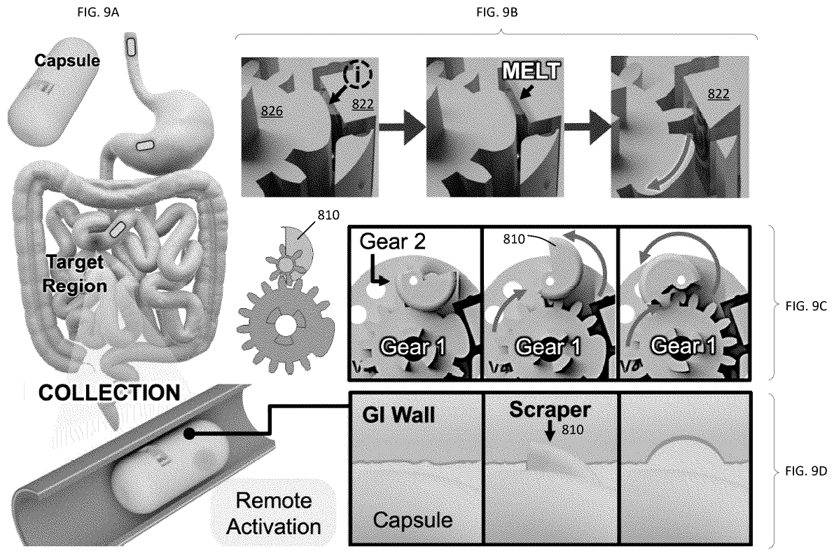

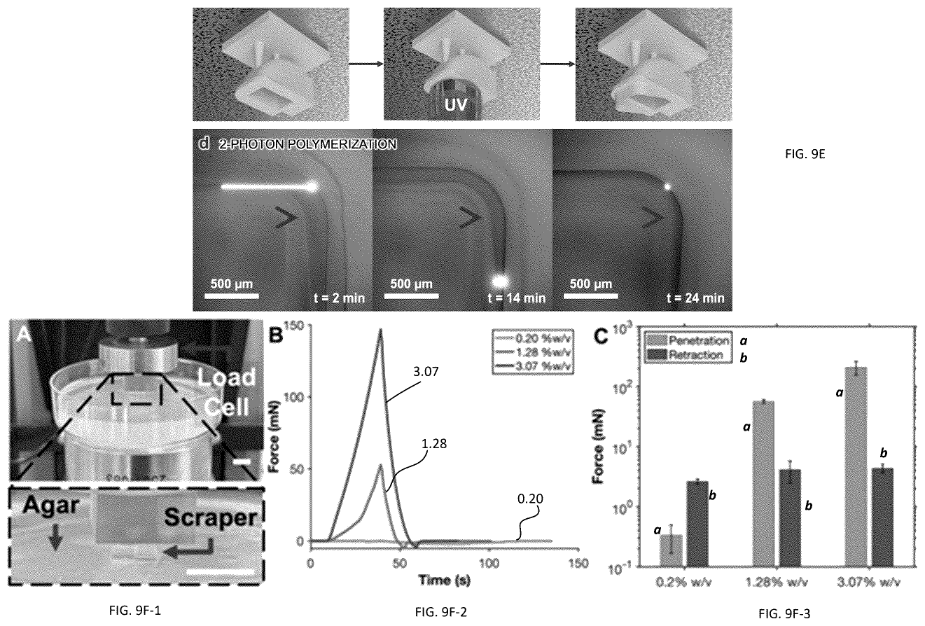

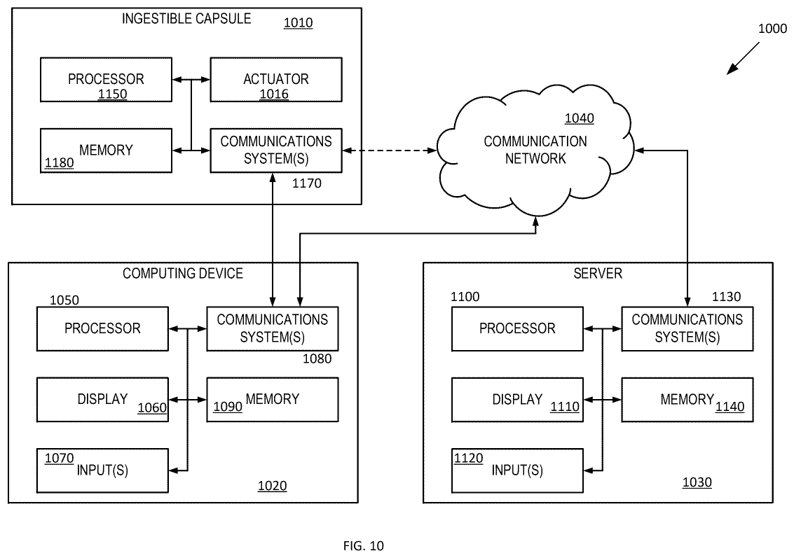

Details of structure and operation of the biopsy module of the embodiment of FIGS. 8A and 8B and now discussed in reference to FIG. 8F, which depicts most of constituent elements of the biopsy module in expanded view. A biopsy collection member of the biopsy module structured to include a rotatable cavity or cup or scoop or scraper 810 having a bladed rim or blade or edge 810A, such that upon rotation of the cavity 810 initiated by the actuation mechanism 818 a portion of GI tissue is severed by the rim and retained within the volume of the rotatable cavity. (As also shown in FIGS. 9A, 9B, 9C, and 9D, providing additional illustrations of the operation of the biopsy module of FIGS. 8A-8C, the actuator mechanism in such embodiment generally includes rotational members aligned with a biopsy collections member so as to cause a movement of the member 810 transversely or substantially transversely to a longitudinal axis of the embodiment of the capsule, into the surrounding GI tissue to extract a sample.)

In particular, the actuation mechanism shown as 818 employs a gear train system to achieve mechanical advantage and control of the biopsy collection member 810. The gear train system includes a base structure 822 that serves as a mounting platform for the other components of the mechanism 818. A first gear 826 (gear 1) of the gear trains system includes a spring slot 826A dimensioned to interface with a torsion spring 830 and features a peripheral portion 826B lacking gear teeth—which portion 826B that is specifically designed to be brought in contact with and bonded to the thermally responsive adhesive 834 (which is disposed between the portion 826B and the microheater 838 of the mechanism 818. (Alternatively, portion 826B may be thought of as a single mega-tooth of the gear 826, dimensioned to have a width that is substantially larger than a width of any other tooth of the gear 826 and for that reason not meshing with a corresponding second gear—gear 846, shown as gear 2—of the gear train.) The second gear 846 is mechanically coupled to a scoop-shaped biopsy collection member 810. When assembled, the heater element 838 is positioned beneath the toothless portion 826B of gear 826, with the thermally responsive adhesive 834 applied between these components. A torsion spring 830 is installed with one end fixed to the base 822 and the other end inserted into the spring slot 826A of gear 826, thereby storing mechanical energy when the gear 826 is rotated to a loaded position.

In one instance, the gear train system of the actuation mechanism such as mechanism 818 may be fabricated with biocompatible polymers such as polyoxymethylene (POM) with a tooth module of 0.3 mm to 0.5 mm. The gears may be mounted on stainless steel pins with diameters between 0.5 mm and 1.0 mm, and may incorporate low-friction bushings made from polytetrafluoroethylene (PTFE). In some cases, the gear teeth may be designed with an involute profile and a pressure angle of 20 degrees.

The rotatable scoop-shaped biopsy member 810 in one case may be fabricated from medical-grade stainless steel or a rigid biocompatible polymer such as polyetheretherketone (PEEK), and dimensioned to have an outer diameter of 2-4 mm, with an internal volume sufficient to store the collected tissue sample. The scoop-shaped member 810 may be manufactured using precision micro-machining techniques with tolerances of ±0.01 mm, incorporating a sharpened leading edge with a cutting angle between 15 and 45 degrees. The cavity/scoop-shaped member 810 may be structured to incorporate a low-friction bearing interface to ensure smooth rotation with respect to the second gear 846 during the tissue sampling process. Alternatively or in addition, the member 810 may include stabilizing features with clearances of 50-100 microns to maintain proper spatial alignment during rotation while preventing unwanted movement. The rotatable cavity design enables precise control over the tissue sampling process while maintaining a compact form factor suitable for an ingestible device.

The bladed rim 810A may be sharpened to an angle of 15-30 degrees to facilitate clean tissue cutting. In one specific case, the torsion spring may be fabricated from stainless steel wire with a diameter of 0.1-0.3 mm and a spring constant of 0.1-0.5 N·mm/degree to provide sufficient torque to drive the gear train when released. The gear train is preferably—but not necessarily—designed with a mechanical advantage ratio between 5:1 and 20:1 to enable the actuator mechanism 818 to operate with power consumption below 200 milliwatts while generating forces exceeding 2.0 Newtons at the biopsy collection tool 810. In some examples, the bladed rim may 810A incorporate serrated edges (not shown) with tooth heights of about 50-200 microns to enhance tissue cutting efficiency.

In reference to FIG. 9E (which includes several illustrations addressing the process of fabrication), the gears, base, and shell of the embodiment 800 were fabricated using biocompatible surgical guide resin through LCD vat photopolymerization (LCD-VPP). Due to the 22 μm X and Y resolution of standard LCD-VPP, achieving sharp, single-micron features is difficult. Therefore, to fabricate the tissue scraper, the scraper base was printed with LCD-VPP, then the scraper blade was printed directly onto the base with UpPhoto utilizing DLW. Both the UpPhoto resin used for the blade and Surgical Guide resin in the base are ISO 10993 compliant materials and non-cytotoxic. FIG. 2G shows the printing process of the blade, where the laser scans over the resin, creating each layer of the structure. Each successive printed layer is made narrower, forming the pyramidal shape of the blade that tapers to a sharp point. During the print, the laser power was set to 50 mW for infills at a scan rate of 600 mm/s throughout the print, with the voxel height of 8.87 μm and voxel width of 0.735 μm. When printing shape outlines or finer features, the laser power and scan rate are modulated to 32 mW and 100 mm/s respectively. At 2- and 14-min print time, the laser can be observed creating the base and mid-section of the blade with infills at widths of 130-200 μm. At 24 min, nearing the zenith of structure, features are likely produced by multiple outlines as the feature width is reduced to <10 μm.

### (I.2) Ensuring Sufficient Pressure at the Target Tissue with a Biopsy Collection Member

A skilled person is aware that one of major challenges to successful application of electromechanical biopsy modules of related art is generating enough pressure at the interface of the GI tissue to effectively and safely sever tissue samples. This is largely due to manufacturing limits in creating sharp and mechanically robust structures for cutting. Literature has shown (see, for example, Barducci, L., et al., Fundamentals of the gut for capsule engineers. Prog. Biomed. Eng. 2, 042002, 2020) that to cut small intestinal tissue, a destructive stress of about 1.2 MPa must be applied throughout contact where thickness of the cutting edge is inversely proportional to the minimum force required for penetration, and higher sharpness of the cutting edge reduces the force necessary to induce destructive stress and penetration. Conventionally, tissue collection structures are fabricated from metals utilizing subtractive machining techniques achieving thicknesses of 30-100 μm. Moreover, the 19-25 G hollow needles traditionally used for fine needle aspiration biopsy have wall thicknesses of ~100-200 μm. The sharpness of the capsular biopsy collection tools reported in literature was about 30 μm (see Le, V. H., et al., Shape memory alloy-based biopsy device for active locomotive intestinal capsule endoscope. Proc. Inst. Mech. Eng. [H] 229, 255-263, 2015), while typically surgical cutting tools feature a tip sharpness of 5-15 μm (see M McCarthy, C. T., et al., On the sharpness of straight edge blades in cutting soft solids: Part I—indentation experiments. Eng. Fract. Mech. 74, 2205-2224, 2007; or Giovannini, M., Ren, H., Wang, X. & Ehmann, K. Tissue Cutting With Microserrated Biopsy Punches. J. Micro Nano-Manuf. 5, 2017). To achieve performance that is advantageously gainful over that of related art, the blade 810A of the scraper 810 was fabricated from a photopolymer resin with a flexural strength of 127 MPa and maximum compressive strength of 132-142 MPa. A bevel angle of 15° was chosen for the blade to reduce drag throughout penetration. The pyramidal cross-sectional design of a body of the scraper 810 was judicially chosen to support structural stability and reduce bending due to moments applied at the blade tip during actuation.

To assess the sharpness of the fabricated scraper blade, scanning electron microscopy (SEM) was conducted on scraper samples before final assembly. Evaluation of the micrographs revealed that the blades had a sharpness corresponding to the blade thickness of approximately 1 μm. Given this thickness and the maximum expected vertical distance, the scraper 810 in practice travels outside of the housing capsule for less than 1 mm, while the initial area of contact of the tissue scraper with the tissue can be estimated to be 0.25 mm². The scraper blade 810A was printed 100 μm into the LCD-VPP 3D-printed base, ensuring a stable bond and fusion of the two materials, as is shown in the micrographs of the blade-base interface.

To validate the mechanical robustness of the scraper blade 810A and its ability to penetrate tissue of different stiffness, the blade was tested on agarose phantoms of varying stiffness. Phantoms were fabricated with concentrations of 0.2, 1.28, and 3.07 % w/v to simulate intestinal mucosa (E=2.6 kPa), inflamed tissue (E=16 kPa), and tumor tissue stiffness (E=40 kPa), respectively. Samples were translated by a mechanical tester at a crosshead speed of 1 mm/min into the phantom until the blade reached full penetration (~500 μm) and retracted until full separation was achieved (see FIG. 9F-1). Penetration forces scaled with the increase in phantom concentration (FIG. 9F-2). The maximum penetration forces of the scraper (FIG. 9F-3) were assessed to be 0.333±0.16, 56.7±3.7, 208±53 mN for 0.2, 1.28, and 3.07 % w/v phantoms respectively; while the retraction forces were 2.62±0.26, 4.13±1.6, 4.36±0.73 mN. While there was a significant increase in the penetration force between the inflamed and tumor tissue phantom penetration, the relative increase in retraction force observed was less than 2×.

Overall, in some embodiments of a biopsy module of an ingestible capsule device (in a non-limiting case of the device 800, for example), the activation mechanism may be structured to ensure that the force exerted on the GI tissue by the biopsy member 810 be greater than 2 Newtons (2 N), and more preferably greater than 2.1 N, or than 2.2 N, or than 2.3 N, or than 2.4 N. With the use of the heating element 838, the power delivered from the source 114 and utilized to cause such force may be as low as 200 mW, or lower than 175 mW, or lower than 150 mW, or even lower depending on the specifics of a particular implementation. In one specific example, the ingestible device is devised such that the biopsy collection member, in operation, applies at least 0.9 MPa of pressure on the GI tissue while consuming approximately 180 mW of power or less from the power source.

The skilled person will now readily appreciate that—as compared with embodiments of the biopsy modules of related art—the proposed implementation achieves a substantial mechanical advantage at the expense of a relatively small input power to generate a significant force for driving the biopsy member into the target GI tissue.

### (I.3) Example of a Tissue Collection Process

Using embodiment of FIGS. 8A, 8B as a non-limiting example, and referring to FIGS. 9A, 9B, 9C, and 9D, the tissue collection process begins when an embodiment of the ingestible capsule reaches a target location in the GI tract (FIG. 9A). At this point, the heater element 838 is activated, applying heat to the thermally responsive adhesive that bonds gear 826 to the base 822. As the adhesive melts, the stored energy in the torsion spring is released, causing gear 826 to rotate. This rotating gear 826 engages with and drives gear 846, which in turn rotates the attached scoop-shaped biopsy collection member 810. The scoop 810 extends through an opening (aperture) in the capsule housing 110 (not shown), rotating approximately 180 degrees to scrape and collect tissue from the intestinal wall. The rotational motion of gear 846 and the attached scoop 810 allows the sharpened edge to effectively separate tissue from the mucosal layer and collect it within the scoop's internal volume. After tissue collection, the scoop 810 continues its rotation to return to the inner volume of the capsule housing, thereby storing the collected sample for later retrieval and analysis. The gear train design ensures that sufficient force is applied during the tissue collection process while maintaining control over the motion path of the collection tool.

Generally, the biopsy collection member 810 may take a variety of forms. In one example, the biopsy collection member may resemble a two-piece nested cup in which an upper cup-shaped member has a sharpened edge. Upon rotation of the cup, it extends outwardly from the capsule to come into contact with, sever and store the tissue in an internal volume suitable for clinical analysis. In another embodiment, the biopsy collection member may comprise a biopsy punch having a cylindrical shape defining an internal, hollow volume. The punch may be actuated so that it comes into forcible contact with the GI tissue, and then retracted along a same axis of movement to drawn the acquired biopsy sample into the capsule for storage. Notwithstanding the form of the biopsy collection member, preferred embodiments of the ingestible device provide that upon actuation, the biopsy collection member and its internal volume defined within the tissue-collection cavity or chamber are located in a first position outside the device housing to penetrate the tissue and collect the tissue sample. Retraction of the biopsy collection member relocates the biopsy collection member and its internal volume in a second position within the device housing for storage of the collected tissue.

### II. Governing the Operation of an Ingestible Capsule Device

FIG. 10 illustrates a schematic of implementation of the overall apparatus or system 1000 governing and/or utilizing the operation of an embodiment of the ingestible capsule device 1010. The apparatus or system 1000 may include, in addition to the ingestible capsule 1010, a computing device 1020, a server 1030, and a communication network 1040 that is configured to enable of data between the computing device 1020 and the server 1030. Various embodiments of the ingestible capsule have been addressed above.

Electronic circuitry of the ingestible capsule 1010 (see FIGS. 1A-1C) may contain a controller comprising a processor 1150 operatively connected to a non-transitory tangible memory 1180. The processor 1150 may be implemented using a low-power microcontroller such as an ARM Cortex-M0+ operating at 16 MHz with 32 KB of flash memory and 8 KB of RAM. The memory 1180 may include both volatile and non-volatile memory components, such as SRAM for temporary data storage and EEPROM for long-term data retention. For example, the memory 118 may utilize a 256 KB EEPROM chip with SPI interface for storing sensor data and device configuration parameters. These electronic components are arranged similarly to the onboard electronics shown in FIGS. 1A-1C, which illustrate the integration of the PCB and battery with the biopsy module.

The controller of the ingestible capsule may be operatively connected to an actuator mechanism (here, 1160) and a communication system 1170. Various implementations of the actuator mechanism 1160 have been already alluded to above and are structured to enable various mechanical functions within the capsule.

The communication system 1170 of the ingestible capsule is configured to facilitate data transmission through the network 1040. For instance, the communication system 1170 may utilize a Bluetooth Low Energy (BLE) transceiver chip operating in the 2.4 GHz ISM band with a maximum transmit power of 0 dBm and a receiver sensitivity of −93 dBm. This wireless capability enables the remote triggering functionality via antenna depicted in FIG. 1C, where the biopsy module can be activated based on sensor feedback or external commands (thus providing for the autonomous operation of the embodiment of the ingestible device).

The computing device 1020 is preferably structured to include a processor 1050 connected to a display 1060, an input interface 1070, an internal communication system 1080, and a memory storage 1090. For example, the processor 1050 may be implemented using a quad-core ARM Cortex-A53 processor operating at 1.4 GHz. The computing device 1020 in operation receives and processes data from the ingestible capsule 1010 through the communication network 1040. If the ingestible capsule includes the optional pH sensor (see FIG. 1A), the computing device 1020 is preferably configured to monitor the pH data collected by the pH indicator/sensor, thereby allowing for real-time tracking of the capsule's location within the GI tract based on environmental pH levels.

The server 1030 may include a corresponding server processor 1100 connected to a display 1110, an input interface 1120, a server communication system 1130, and a memory storage 1140. For instance, the server 1030 may utilize a multi-core Intel Xeon processor with 64 GB of RAM and 1 TB of solid-state storage. The server 1030 may be configured to connect to both the computing device 1020 and the ingestible capsule 1010 through the communication network 1040.

In some examples of operation of the apparatus 1000, a subject may ingest the capsule 1010 in a hospital or other medical setting. In this scenario, the computing device 1020 may be a dedicated medical workstation equipped with specialized software for real-time monitoring of the capsule's progress through the gastrointestinal tract. The workstation may be connected to the hospital's secure local area network, allowing healthcare professionals to access and analyze the data collected by the capsule 1010. The capsule's biocompatible housing ensures safe passage through the GI tract while the internal components, including an embodiment of the activation mechanism, enable precise mechanical actuation for tissue sampling.

The apparatus 1000 may additionally incorporate various security measures to protect sensitive medical data. For example, all data transmissions between the capsule 1010, computing device 1020, and server 1030 may be encrypted using AES-256 encryption. Additionally, the server 1030 may implement multi-factor authentication and role-based access control to ensure that only authorized personnel can access patient data. This secure communication framework supports the complete operational sequence of the embodiment of the ingestible capsule, from initial ingestion through tissue sampling using various mechanisms and methodologies discussed above, to provide for relevant data analysis and storage.

### III. Additional Considerations

The skilled person will readily appreciate that various structural and operational deviations from the embodiments already discussed can be implemented in practice.

### (III.A) Examples of Variations on Activating Mechanism

For example, the extension of the punch 226 (discussed in reference to FIGS. 3A-3D, 4A-4C) may be driven by a miniature linear actuator with a stroke length of 5-10 mm and a force output of 1-to-2 N. The biomimetic collector structures may then engage with the tissue, allowing for controlled sampling. The linear actuator may utilize a shape memory alloy (SMA) wire such as nickel-titanium alloy with a diameter of 50-100 microns and a transition temperature of 37-40 °C to generate the required force and displacement when electrically heated. In some examples, the actuator may incorporate a mechanical advantage mechanism such as a lever system with a ratio of 3:1 to 5:1 to amplify the force generated by the SMA wire. The actuation system may be controlled by a microcontroller such as an ARM Cortex-M0+ operating at 16 MHz with current-limiting circuitry to prevent overheating of the SMA wire. For example, the actuation sequence may be programmed with a gradual extension profile over 1-2 seconds to allow tissue to accommodate to the insertion force, followed by a rapid retraction over 0.2-0.5 seconds to cleanly sever the tissue sample.

Alternatively, an actuation mechanism (such as that employing the gear train discussed above) may be structured with a mechanical advantage ratio between 5:1 and 20:1 and/or be alternatively coupled with—instead of the torsion spring-a miniature DC motor with a diameter of 4 mm to 6 mm that operates at about 3 V with a no-load speed within a range from about 8000 rpm to about 12000 rpm and a stall torque of 0.1 mN·m to 0.3 mN·m. Here, the gear train may be fabricated using injection-molded polyoxymethylene (POM) plastic with a tooth module of 0.3 mm to 0.5 mm and a pressure angle of 20 degrees. For example, the gears may be mounted on stainless steel pins with diameters between 0.5 mm and 1.0 mm, incorporating low-friction bushings made from polytetrafluoroethylene (PTFE). In some cases, the gear teeth may be designed with an involute profile. Generally, the power consumption of such specific actuator mechanism may be lower than about 150 milliwatts during biopsy operation. This low power consumption may be achieved through, for example, using coreless motor design, with a rotor constructed from a copper winding supported by a polymer frame. The motor may incorporate neodymium magnets with a remanence of 1.2 T to 1.4 T. In some examples, the motor may be controlled using a pulse-width modulation (PWM) signal generated by a microcontroller, with a frequency between 20 kHz and 50 kHz. The motor driver circuitry may incorporate low-resistance MOSFETs with RDS(on) values below 50 mΩ. For instance, the control system may implement current limiting using a sense resistor with a value between 0.1 Ω and 1.0 Ω connected to a comparator circuit with a response time of less than 1 μs.

In operation of the embodiment in which the activation mechanism employs a microheater, when the microheater is activated, the meltable polymer releases the torsion spring, initiating the actuation sequence. The released energy from the torsion spring drives the gear train, which in turn rotates the cam. As the cam rotates, the follower translates the rotational motion into linear displacement, extending the biopsy collection member out of the capsule housing. After tissue collection, the foam spring assists in retracting the biopsy collection member back into the capsule housing, completing the sampling cycle. For example, the actuation sequence may be controlled by a microcontroller such as an ARM Cortex-M0+ operating at 16 MHz with 32 KB of flash memory and 8 KB of RAM. The microcontroller may execute firmware that implements a state machine to manage the actuation timing, with states for initialization, heating, deployment, sampling, retraction, and completion. In some cases, the microcontroller may monitor the current drawn by the microheater to detect when the polymer has melted, using a current sensing resistor with a value of 1 Ω to 10 Ω and an analog-to-digital converter with 12-bit resolution. The firmware may implement a timeout mechanism to prevent battery depletion if the actuation sequence fails to complete, automatically shutting down the microheater after a predetermined period of 5 seconds to 10 seconds.

### (III.B) Examples of Variations on Anchoring Mechanism

In some cases, an anchoring mechanism may be included to removably affix the device to a location of interest within the GI tract. This mechanism may take various forms, such as expandable structures that temporarily grip the intestinal walls or bio-adhesive patches that provide temporary attachment. For instance, the anchoring mechanism may utilize hydrogel-based adhesives that bond to mucosal surfaces but degrade over time to allow eventual passage of the device. In some examples, the anchoring mechanism may be implemented using a shape memory alloy framework that expands to a predetermined diameter between 15 mm and 30 mm when deployed in the intestine. In some implementations, the anchoring mechanism may incorporate microdarts fabricated from biodegradable polymers such as polylactic acid (PLA) or polyglycolic acid (PGA) with lengths between 0.5 mm and 2.0 mm. The anchoring mechanism may withstand peristaltic forces of up to 0.3 N while maintaining position within the GI tract for periods ranging from 1 hour to 24 hours, depending on the specific application requirements.

### (III.C) Examples of Variations on the Control System

The control systems may utilize low-power wireless protocols such as Bluetooth Low Energy (BLE) for communication with external receivers. For example, the wireless communication may operate in the 2.4 GHz ISM band with a maximum transmit power of 4 dBm and receiver sensitivity of −96 dBm. The PCB may incorporate power management integrated circuits such as buck converters to efficiently regulate voltage from the power source to the required levels for digital and analog circuitry. For instance, the power management system may include a low-dropout regulator with an output voltage of 1.8V to 3.3V and a quiescent current below 1 μA to power the microcontroller and sensor interfaces. The wireless transceiver may utilize a chip antenna with dimensions of 3.2 mm×1.6 mm and a gain of −1 dBi to 2 dBi, or a printed circuit board antenna with a meandered trace pattern occupying an area of approximately 5 mm×10 mm. In some examples, the wireless protocol stack may implement adaptive frequency hopping with 37 channels.

In some cases, the control systems may implement duty cycling with active periods of 50 ms to 200 ms followed by sleep periods of 1 s to 10 s. The firmware executed by the microcontroller may implement a state machine to manage the actuation timing, with states for initialization, heating, deployment, sampling, retraction, and completion. For instance, the microcontroller may monitor the current drawn by the microheater to detect when the polymer has melted, using a current sensing resistor with a value of 1 Ω to 10 Ω and an analog-to-digital converter with 12-bit resolution. The firmware may be developed using a real-time operating system (RTOS) such as FreeRTOS or Zephyr with a memory footprint of 8 KB to 32 KB to manage concurrent tasks and timing requirements. For example, the firmware may implement a watchdog timer with a timeout period of 1 s to 10 s to reset the system in case of software failures. In some examples, the firmware may store operational data and sensor readings in non-volatile memory such as EEPROM or flash memory with capacities of 32 KB to 256 KB, using wear-leveling algorithms.

Overall, the overall design of the ingestible biopsy device may prioritize miniaturization and power efficiency to enable extended operation within the GI tract. Advanced manufacturing techniques such as 3D printing or micro-molding may be employed to create intricate internal structures and mechanisms within the compact housing. The integration of these various components and functionalities into a single ingestible form factor may enable non-invasive, targeted tissue sampling in regions of the GI tract. For example, the device housing may be fabricated using digital light processing (DLP) 3D printing with biocompatible resins such as dental SG resin. Internal mechanical components may be manufactured using micro-injection molding of medical-grade polymers such as PEEK or POM. The device may incorporate a system-on-package design approach where multiple semiconductor dies are integrated within a single package using wire bonding or flip-chip techniques. In some examples, power management circuitry may implement duty cycling with active periods followed by sleep periods.

### (III.D) Examples of Variations on Sensing Capabilities of an Embodiment of the Device

The ingestible capsule biopsy device may perform multiple functions beyond tissue sampling. For example, the device may incorporate additional sensing capabilities, allowing for targeted monitoring at specific locations in the GI tract. This may be achieved through the inclusion of various sensors controlled by the onboard electronics.

To enhance its diagnostic capabilities, the device may integrate various sensors to monitor GI environmental parameters. These may include pH sensors, temperature probes, or chemical sensors to detect specific biomarkers. For example, miniaturized ion-selective field-effect transistors (ISFETs) may be used to measure local pH levels, providing contextual information for tissue sampling decisions. The device may incorporate one or more electrochemical sensors configured to monitor GI tract tissue characteristics and determine the area of interest based on detected electrochemical signals. For instance, the pH sensors may utilize iridium oxide sensing elements with a measurement range from pH 2 to pH 9 and accuracy of ±0.1 pH units. For example, temperature sensing may be implemented using miniature negative temperature coefficient (NTC) thermistors with a measurement range from 25 °C to 45 °C and accuracy of ±0.2°C. In some implementations, the device may include amperometric sensors for detecting inflammatory biomarkers such as myeloperoxidase or calprotectin, utilizing screen-printed carbon electrodes modified with specific antibodies or aptamers.

An embodiment of the device may include a pH indicator/sensor with the onboard electronics. The pH sensor may be implemented using ion-selective field-effect transistors (ISFETs) with a measurement range from pH 2 to pH 9. For instance, the pH-responsive coating may be applied to a gold pad/lead using techniques such as electrodeposition or spin-coating. The pH sensor may incorporate a reference electrode fabricated from silver/silver chloride (Ag/AgCl) to provide a stable potential reference. For example, the sensor interface circuitry may include an operational amplifier configured as a voltage follower with an input impedance and an offset voltage. In some cases, the pH sensor may be calibrated using a two-point calibration procedure with standard buffer solutions of pH 4.0 and pH 7.0 to establish the relationship between sensor output and pH value.

The ingestible device may incorporate electrochemical sensing capabilities to enable real-time monitoring of biomarkers in the gastrointestinal (GI) tract. The device may include a three-electrode sensor comprising a working electrode, counter electrode, and reference electrode fixed to the outer surface of the capsule housing. The electrodes may be fabricated using photolithography techniques with gold traces on a flexible polyimide substrate. The device may incorporate sensors for detecting various biomarkers that could trigger the biopsy mechanism, including: serotonin sensors using gold-carbon nanotube composite electrodes; C-reactive protein sensors utilizing antibody-modified electrodes; calprotectin sensors employing aptamer-functionalized surfaces; lactoferrin sensors based on immunoelectrochemical detection; myeloperoxidase sensors using screen-printed carbon electrodes modified with specific antibodies; and bioimpedance sensors to detect changes in tissue permeability associated with inflammation. The device may also incorporate cytokine sensors (IL-6, TNF-α) using sandwich immunoassay configurations on gold nanoparticle-modified electrodes, and bacterial metabolite sensors for detecting short-chain fatty acids using pH-responsive polymers coupled with potentiometric measurement. These sensors could be selectively activated based on location within the GI tract, as determined by pH measurements, to conserve power and provide targeted biomarker detection.

Referring again to FIG. 1A, for example, in a specific embodiment of the ingestible capsule equipped with a pH indicator/sensor that is integrated with the onboard electronics 118, the pH sensor 126 may be implemented using ion-selective field-effect transistors (ISFETs) with a measurement range from pH 2 to pH 9 and accuracy of about ±0.1 pH units. For instance, the pH-responsive coating may be applied to a gold pad/lead using techniques such as electrodeposition or spin-coating, with layer thicknesses ranging from 100 nm to 1 μm. The pH sensor may incorporate a reference electrode fabricated from silver/silver chloride (Ag/AgCl) with dimensions of 1 mm×1 mm and a thickness of 200 nm to 500 nm to provide a stable potential reference. For example, the sensor interface circuitry may include an operational amplifier configured as a voltage follower with an input impedance greater than 10 GΩ and an offset voltage below 1 mV. In some cases, the pH sensor may be calibrated using a two-point calibration procedure with standard buffer solutions of pH 4.0 and pH 7.0 to establish the relationship between sensor output and pH value.

### (III.E) Preservation of Collected Tissue Samples

To preserve the already collected tissue samples, the internal volume of the biopsy punch may be pre-loaded with a small quantity (e.g., 10-50 microliters) of a tissue fixative such as jaggery syrup. This fixative may help maintain the structural and molecular integrity of the sample during device transit through the GI tract. In some cases, the device may include a microfluidic system to deliver preservative solutions to the sample chamber after collection. For instance, this system may utilize a series of microchannels with widths of 100-500 microns, fabricated using soft lithography techniques in polydimethylsiloxane (PDMS). The microfluidic system may incorporate passive capillary valves with sudden channel expansions from 100 microns to 300 microns to control fluid flow without requiring active components. For example, the preservative reservoir may utilize a compressed elastomer design with a silicone membrane of 200-500 microns thickness under 10-30% compression to provide the pressure gradient for fluid delivery. In some examples, the microfluidic channels may be treated with oxygen plasma at 50-100 W for 30-60 seconds to render the PDMS surface hydrophilic with contact angles below 20 degrees. The preservative delivery system may incorporate a one-way valve mechanism using a ball-and-seat design with a ruby sphere of 300-500 microns diameter and a precision-machined seat with a conical profile of 60-90 degrees.

The effectiveness of different collector designs may be evaluated based on factors such as sample volume, tissue retention, and preservation quality. For example, experimental testing may involve deploying the various collector designs into tissue phantoms or ex vivo tissue samples, followed by quantitative analysis of collected sample mass and histological assessment of tissue integrity. In some cases, the hooked barb design may demonstrate superior tissue retention, with sample masses 2-3 times greater than smooth-walled collectors. The sawtooth ring structures may provide a balance between sample volume and preservation, as the multiple cutting surfaces may help maintain tissue structure during collection. The tissue phantoms used for testing may be fabricated from polyacrylamide gels with concentrations of 5-10% and elastic moduli of 10-50 kPa to mimic the mechanical properties of intestinal mucosa. For instance, ex vivo tissue testing may utilize porcine intestinal samples preserved in phosphate-buffered saline at 4 °C and used within 24 hours of harvesting to maintain tissue properties similar to in vivo conditions. In some examples, the collected samples may be evaluated using histological techniques including hematoxylin and eosin staining with section thicknesses of 5-10 microns to assess tissue morphology and integrity. Quantitative analysis may include measurements of sample mass using microbalances with resolutions of 0.01 mg, sample volume determination using optical coherence tomography with axial resolutions of 5-10 microns, and assessment of cellular viability using metabolic assays such as MTT with colorimetric readouts at 570 nm wavelength.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,”“in an embodiment,”and similar language throughout this specification may, but do not necessarily, all refer to the same embodiment. It is to be understood that no portion of disclosure, taken on its own and in possible connection with a figure, is intended to provide a complete description of all features of the invention.

Within this specification, embodiments have been described in a way that enables a clear and concise specification to be written, but it is intended and will be appreciated that embodiments may be variously combined or separated without parting from the scope of the invention. In particular, it will be appreciated that all features described herein at applicable to all aspects of the invention.

In addition, when the present disclosure describes features of the invention with reference to corresponding drawings (in which like numbers represent the same or similar elements, wherever possible), the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. Other specific examples of the meaning of the terms “substantially”, “about”, and/or “approximately” as applied to different practical situations may have been provided elsewhere in this disclosure. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The term of the type “A and/or B” Is Defined to Mean, Cover, and Represent Each of “A”, “B”, and “A and B”

While the invention is described through the above-described examples of embodiments, it will be understood by those of ordinary skill in the art that modifications to, and variations of, the illustrated embodiments may be made without departing from the inventive concepts disclosed herein. Furthermore, disclosed aspects, or portions of these aspects, may be combined in ways not listed above. Accordingly, the invention should not be viewed as being limited to the disclosed embodiment(s).

1. An ingestible device for gastrointestinal (GI) tissue sampling, the device comprising:
a housing;
a power source disposed within the housing; and
a biopsy module that includes an actuator mechanism operably coupled to the power source and a biopsy collection member having a cavity defining an internal volume thereof and actuatable by the actuator mechanism to have the cavity reversibly extend outside of the housing,
wherein the device is operable without direct human control.

2. A device according to claim 1 that does not involve magnets and/or magnet-stimulated mechanisms.

3. A device according to claim 1, further comprising a trigger mechanism to trigger the actuation mechanism, the trigger mechanism comprising one or more of the following:
a bioimpedance sensor;
a wireless communication system;
a pH sensor;
an electrochemical sensor;
a timer; and
a biomarker sensor or a temperature sensor.

4. A device according to claim 3, wherein, when the trigger mechanism includes the electrochemical sensor, the electrochemical sensor is operable to monitor a characteristic of the GI tract tissue in real time and to determine an area of interest for monitoring the characteristic based on electrochemical signals registered inside the GI tract.

5. A device according to claim 1, wherein the actuator mechanism comprises a rotational member operated in response to power delivered from the power source, the rotational member being configured to move the biopsy collection member into contact with the GI tract tissue.

6. A device according to claim 5, wherein the actuator mechanism further comprising an initiating member configured to move from a detained position to a released position to apply a rotational force to the rotational member.

7. A device according to claim 6, wherein the actuator mechanism comprises a cam of a cam-and-follower mechanism to convert a rotational motion into a linear displacement of the biopsy collection member.

8. A device according to claim 7, wherein the biopsy collection member is configured as a follower of the cam-and-follower mechanism and the housing is configured as a frame and a guide of the cam-and-follower mechanism.

9. A device according to claim 5, wherein the actuator mechanism comprises a gear train configured to transmit torque from a structural element of the device to the biopsy collection member after a status of the structural element has been modified by the power source.

10. A device according to claim 1, wherein the biopsy collection member comprises i) a biopsy punch having a cylindrical shape or ii) a rotatable cavity, the biopsy collection member having a bladed rim, wherein the internal volume is dimensioned to be approximately 1 mm³ to approximately 5 mm³ to hold a sample of the GI tissue sufficient in size for clinical diagnostic testing.

11. A device according to claim 1, wherein the biopsy collection member is configured to apply at least 0.9 MPa of pressure on the GI tissue while consuming approximately 180 mW of power or less from the power source.

12. A device according to claim 1,
wherein the actuator mechanism comprises a spring, a microheater, and a thermally responsive adhesive,
wherein the spring is configured with one end thereof fixed to the housing and another end thereof attached to a rotatable component of the device,
wherein the microheater is thermally coupled to the thermally responsive adhesive, and wherein the thermally responsive adhesive that is configured to restrain the spring in a loaded state and to release the torsion spring to drive the biopsy collection member when molten or softened by heat energy from the microheater.

13. A device according to claim 12, wherein the spring is connected to and retained with a gear that is directly bonded to a fixed element of the actuation mechanism with the thermally responsive adhesive.

14. An autonomous ingestible device for gastrointestinal (GI) tissue sampling, the device comprising:
a housing;
an on-board power source contained within the housing;
a moveable apparatus coupled with the housing and having a portion that is reversibly repositionable from a retracted position inside the housing to an extended position outside the housing upon autonomous activation by power delivered from the power source;
a thermally responsive adhesive disposed within the housing and configured to retain the biopsy collection member in the retracted position;
a spring disposed within the housing and operatively connected to the portion of the movable apparatus, wherein the spring is in a loaded state when the portion is in the retracted position; and
a heater electrically coupled to the power source and disposed adjacently to the thermally responsive adhesive.

15. An autonomous ingestible device according to claim 14, wherein the portion of the moveable apparatus is configured to move linearly or rotationally.

16. An autonomous ingestible device according to claim 14, wherein the portion of the moveable apparatus is a biopsy collection member that includes a) a biopsy punch having a cylindrical shape, or b) a rotatable cavity, wherein the internal volume is about 1 mm³ to about 5 mm³.

17. An autonomous ingestible device according to claim 16, wherein the portion of the moveable apparatus includes a bladed rim.

18. An autonomous ingestible device according to claim 14, wherein the portion of the moveable apparatus has microscale biomimetic tissue collector structures on an inner surface thereof.

19. An autonomous ingestible device according to claim 18, wherein the biomimetic tissue collector structures comprise:
a) hooked barbs arranged in a helical pattern along the inner surface, or
b) sawtooth barbs arranged in rows on the inner surface, or
c) sawtooth ring structures positioned circumferentially on the inner surface.

20. An autonomous ingestible device according to claims 14, wherein the moveable apparatus includes a gear train and a rotatable cavity affixed to a chosen gear of the gear train, and wherein the thermally responsive adhesive is in contact with a gear of the gear train when the member is in the retracted position.

21. An autonomous ingestible device according to claim 14, wherein the moveable member includes a cam-and-follower mechanism, and wherein the housing is configured to be a frame and a guide of the cam-and-follower mechanism.

22. An autonomous ingestible device according to claim 14, further comprising an antenna and electronic circuitry configured to reversibly activate delivery of power from the power source to the heater in response to an activating signal acquired by the antenna from outside of the ingestible device.

23. A method comprising:
autonomously collecting gastrointestinal (GI) tissue by at least:
actuating a rotational motion of a driving mechanism of an ingestible device that contains a power source inside a capsule housing of the device by delivering a power from the power source to a thermally-responsive retaining element mechanically connected with the driving mechanism; and
with said rotational motion, protruding a biopsy collection member from inside the housing to outside the housing to intersect the GI tissue.

24. A method according to claim 23, wherein said actuating includes applying a rotational force to the driving mechanism with an energy-storing element of the ingestible device upon releasing the energy-storing element from a loaded state.
